(19) United States
(12) Patent Application Publication
(10) Pub. No.: US 2010/0233736 A1
(43) Pub. Date: Sep. 16, 2010

(54) DETECTION METHOD AND KIT

(76) Inventors: Michel Duchene, Rixensart (BE); Didier Giffroy, Rixensart (BE); Christophe Mazy, Rixensart (BE)

Correspondence Address: GlaxoSmithKline;GLOBAL PATENTS -US, UW2220, P. O. BOX 1539, KING OF PRUSSIA, PA, 19406-0939, US

(21) Appl. No.: 12/300,365

(22) PCT Filed: May 11, 2007
(86) PCT No.: PCT/EP2007/054550

§ 371 Date: Nov. 11, 2008

(30) Foreign Application Priority Data
May 15, 2006 (GB) 0609612.7

(51) Int. Cl. G01N 33/53

(52) U.S. Cl. 435/7.92

(57) ABSTRACT

The present invention relates to a method of detection and/or quantification of an antigen linked to a metal salt. The antigen may be present within a mixture of different antigens, such as in a multivalent vaccine composition. The invention further provides a kit for use in the method of the invention.

### BACKGROUND

The present invention relates to detection methods for antigens which are linked to metal salts.

In particular the invention relates to methods for detecting the amount of an antigen which is linked to aluminium salts such as aluminium hydroxide or phosphate or aluminium hydroxyphosphate sulphate.

Prior art methods are known that can be used to detect antigens linked to metal salts in this way, for example see WO2004/038417 and the Abbott Auszyme™ kit, both of which can be used for the analysis of, for example, hepatitis B vaccines which contain aluminium hydroxide.

Hepatitis B vaccines containing purified recombinant Hepatitis B surface antigen (HBsAg) particles are used widely around the world and are increasingly employed as components of routine paediatric combination vaccines. The quality control of these vaccines requires the determination of potency and the majority are now tested using an in vitro potency assay which measures the HBsAg content. Manufacturers have developed assays that are product specific but these are all mainly based on a commercial ELISA kit (Auszyme™ EIA kit from Abbott laboratories) and used by many national control laboratories for official testing.

The Auszyme™ kit is based on a sandwich technique using beads coated with mouse monoclonal IgM anti-HBsAg and mouse monoclonal IgG anti-HBsAg labelled with peroxidase, and it can be used on vaccines without prior desorption of the HBsAg from aluminium adjuvant.

Manufacturers of a Cuban hepatitis B vaccine have developed an indirect method for hepatitis B measurement—see Cuervo and Huerta, Validation of an in vitro potency test for the Cuban hepatitis B vaccine, Dev Biol (Base1). 2002; 111:305-12 and Cuervo et al Biologicals 32, 2004, 171-176.

Methods are still needed for detection of antigens which are linked to metals or metal salts, in particular detection of such antigens in the context of larger antigenic combinations. The present invention addresses this need.

### STATEMENT OF INVENTION

In a first aspect the invention relates to a method for detection and/or quantification of an antigen linked to a metal salt, the method comprising:
1 contacting the antigen with an antibody specific for that antigen;
2 contacting any unbound antibody with an antigen linked to a solid support, under conditions that allow any antibody present to form an antibody-antigen complex with the antigen on the solid support; and
3 detecting the antibody-antigen complex formed on the solid support.

In a related aspect the invention relates to a method for detection and/or quantification of a hepatitis B antigen linked to an aluminium salt, the method comprising
1 contacting the antigen with an antibody specific for that antigen;
2 contacting any unbound antibody with an antigen linked to a solid support, to allow any antibody present to form an antibody-antigen complex with the antigen on the solid support; and
3 detecting the antibody-antigen complex formed on the solid support.

In another related aspect the invention relates to a method for detection and/or quantification of a first antigen linked to a metal salt, the antigen being in combination with at least one other (second) antigen, the method comprising
1 contacting the first antigen with an antibody specific for that first antigen;
2 contacting any unbound antibody with an antigen linked to a solid support, to allow any antibody present to form an antibody-antigen complex with the antigen on the solid support; and
3 detecting the antibody-antigen complex formed on the solid support.

In another aspect the invention relates to a kit for detection and/or quantification of an antigen linked to a metal salt, the kit comprising instructions to carry out the method of the invention and at least one other component selected from:
1 an antibody (detection antibody) specific for an antigen to be detected;
2 an antigen, optionally linked to a solid support;
3 detection reagents for detection of any antibody-antigen complex formed on the solid support;
4 positive control; and
5 negative control.

### DETAILED DESCRIPTION

The present invention relates to an inhibition assay which indirectly measures the content of an antigen in a sample in which the antigen is linked to a metal salt. The method using antibodies (eg polyclonal antibodies) in an ELISA type assay. In summary, antibodies which specifically interact with the antigen of interest are contacted with a sample containing the antigen-metal salt complex. After a suitable time (to allow the antibodies to react with antigen in the sample) any unbound antibody is allowed to react with an antigen bound to a solid support, such as an ELISA plate. Detection of the antibody bound to the antigen on the plate gives an indication of the quantity of unbound antibody arising from the initial antibody-antigen sample reaction. Thus, the more antibody detected by the ELISA method, the more unbound antibody was present after the original antibody-antigen reaction and the lower the quantity of antigen in the original sample.

This approach diminishes any interference due to the presence of a metal component in the ELISA plate.

Thus in a first aspect the invention relates to a method for detection and/or quantification of an antigen linked to a metal salt, the method comprising:

1 contacting the antigen with an antibody specific for that antigen;
2 contacting any unbound antibody with an antigen linked to a solid support, to allow any antibody present to form an antibody-antigen complex with the antigen on the solid support; and
3 detecting the antibody-antigen complex formed on the solid support.

The antigen may be any suitable antigen. In one aspect the antigen is a hepatitis B surface antigen. Experiments have been carried out herein using a hepatitis B antigen, but the invention is applicable to other antigens linked with metal salts (which may be adjuvants). Thus any reference to hepatitis B surface antigen (HBsAg) herein is taken to be a reference to any suitable antigen which may be linked to a metal salt, including specifically HBsAg, unless otherwise apparent from the context.

Metal salts of the invention include aluminium, zinc, iron or calcium salts such as aluminium hydroxide, aluminium phosphate, calcium phosphate, zinc hydroxide or calcium hydroxide.

The term ‘antibody’ can relate herein to, for example, an intact antibody with 2 heavy and light chains, or can relate to any sub-fragment thereof, such as a Fv region, which retains the specific antigenic binding capability characteristic of antibodies and well known in the art.

Antigens may be linked to a metal salt by mixing the antigen with the metal under conditions such that the antigen adsorbs onto the metal salt. Such conditions are well known in the art (see for example Vaccine. 2004 Mar. 29; 22(11-12):1475-9, “Mechanism of adsorption of hepatitis B surface antigen by aluminium hydroxide adjuvant”.)

In one aspect the antibody-antigen complex formed on the solid support is detected using an antibody, in one aspect a labelled antibody. In one aspect the antibody specific for the antibody portion of the antigen-antibody complex, such as the Fc region of the antibody.

The present invention is suitable for assessment of the presence and quantity of antigens found in vaccines, such as hepatitis B surface antigen adsorbed to either aluminium hydroxide, aluminium phosphate or aluminium hydroxyphosphate sulphate, for example.

The meaning of the terms ‘aluminium phosphate’ and ‘aluminium hydroxide’ as used herein thus includes all forms of aluminium hydroxide or aluminium phosphate which are suitable for adjuvanting vaccines.

For example, aluminium phosphate can be a precipitate of insoluble aluminium phosphate (amorphous, semi-crystalline or crystalline), which can be optionally but not exclusively prepared by mixing soluble aluminium salts and phosphoric acid salts. “Aluminium hydroxide” can be a precipitate of insoluble (amorphous, semi-crystalline or crystalline) aluminium hydroxide, which can be optionally but not exclusively prepared by neutralizing a solution of aluminium salts. Particularly suitable are the various forms of aluminium hydroxide and aluminium phosphate gels available from commercial sources for example, Alhydrogel (aluminium hydroxide, 3% suspension in water) and Adju-fos (aluminium phosphate, 2% suspension in saline) supplied by Superfos (Vedbaek, 2950 Denmark).

Thus in a related aspect the invention relates to a method for detection and/or quantification of a hepatitis B antigen linked to an aluminium salt, the method comprising
1 contacting the antigen with an antibody specific for that antigen;
2 contacting any unbound antibody with an antigen linked to a solid support, to allow any antibody present to form an antibody-antigen complex with the antigen on the solid support; and
3 detecting the antibody-antigen complex formed on the solid support.

In one aspect the invention relates to detection of an antigen within a larger mixture of antigens, such as a multivalent vaccine composition. In particular the invention relates to detection of a hepatitis B antigen in a combination of that antigen with one or more antigens selected from the list consisting of diphtheria, tetanus, pertussis, Inactivated Polio (IPV); Haemophilus influenzae b (Hib); and Hepatitis A (HA).

In one aspect the invention relates to the detection of hepatitis B surface antigen in a combination with, for example:
1 hepatitis A antigen;
2 Diphtheria toxoid, Tetanus toxoid, Pertussis toxoid, Filamentous haemagglutinin, Pertactin, Poliovirus type 1 (eg Mahoney), Poliovirus type 2 (eg MEF-1), Poliovirus type 3 (eg Saukett);
3 Diphtheria toxoid, Tetanus toxoid, Pertussis toxoid, Filamentous haemagglutinin, Pertactin;
4 Diphtheria toxoid, Tetanus toxoid, Whole cell B. pertussis antigens;
5 H. influenzae type b polyribosyl ribitol phosphate conjugated to tetanus toxoid.

In one aspect the hepatitis B is adsorbed to aluminium phosphate when in the context of combination vaccines containing multiple antigens. See for example WO93/24148, incorporated herein by reference.

Thus in one aspect the invention relates to a method for detection and/or quantification of a first antigen linked to a metal salt, the antigen being in combination with at least one other (second) antigen, the method comprising
1 contacting the first antigen with an antibody specific for that first antigen;
2 contacting any unbound antibody with an antigen linked to a solid support, to allow any antibody present to form a complex with the antigen on the solid support; and
3 detecting the antibody-antigen complex formed on the solid support.

In a further aspect of the invention the method for detection and/or quantification comprises an additional method step between steps 1 and 2 of the methods above, in which the unbound antibody from step 1 is separated from the bound antibody before step 2 is carried out. Separation may be carried out using techniques well known in the art, such as centrifugation, for example as described in the examples herein.

In another aspect the invention relates to a kit for detection and/or quantification of an antigen linked to a metal salt.

In one aspect the kit comprising instructions to carry out the method of the invention and at least one other component selected from:
1 an antibody (detection antibody) specific for an antigen to be detected;
2 an antigen, optionally linked to a solid support;
3 detection reagents for detection of any antibody-antigen complex formed on the solid support;
4 positive control; and
5 negative control.

In one aspect the kit comprises a plate pre-coated with antigen and an antibody, preferably labelled, for specific binding to the detection antibody. In one aspect the plate is coated with hepatitis B surface antigen.

In one aspect the antibodies used are human polyclonal antibodies, such as anti-HBs antibodies (Nabi-HB™—Nabi Biopharmaceuticals, Boca Raton, Fla. USA) in the case of hepatitis B detection.

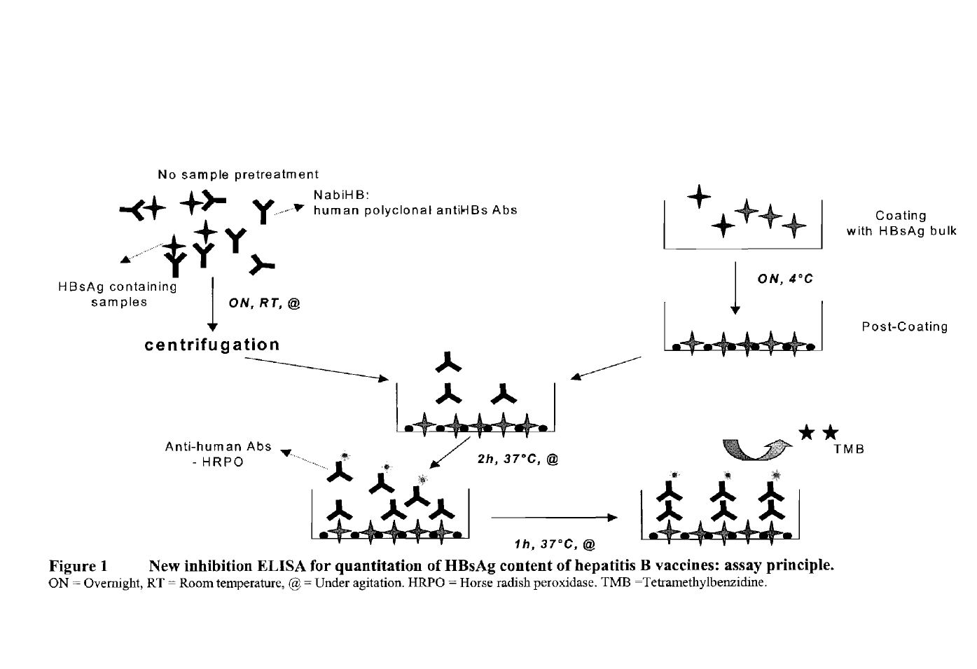

An overview of the method of the invention is provided in FIG. 1, and the method can be generally applied to analysis of antigen linked to metal salts.

The present invention is illustrated by the following example, which is not limiting upon the present invention.

### Example 1

The discontinuation of the Auszyme™ kit used by manufacturers and national control laboratories to determine the Hepatitis B surface antigen (HBsAg) content of hepatitis B vaccines has led us to develop an alternative inhibition ELISA method.

Validation studies were performed according to The International Conference of Harmonization and reproducibility was assessed in a feasibility study with four Official Medicine Control Laboratories (OMCLs).

The dose response curve demonstrated linearity (R²>0.99) in the range of 60-360 ng/ml HBsAg. The repeatability (CV<7%), intermediate precision (CV<10%) and accuracy (91-113% recovery) were similar to the Auszyme™ method. The commercial antibodies used in the assay were shown to bind to a protective epitope of HBsAg and the specificity of the method for HBsAg was demonstrated. There was a good concordance with the Auszyme™ method although the ELISA yielded higher results (25.3 vs. 24.4 μg/ml for Engerix™-B (n=64), 28.9 vs. 27.0 μg/ml for Twinrix™ (n=69) and 25.5 vs. 21.6 μg/ml for Infanrix™ penta (n=62)). The method was successfully transferred to the four OMCLs.

We have demonstrated that the ELISA is suitable for its intended purpose and to be considered as a common method for worldwide use by national control laboratories and authorities.

### 1. INTRODUCTION

Hepatitis B vaccines containing purified recombinant Hepatitis B surface antigen (HBsAg) particles are used widely around the world and are increasingly employed as components of routine paediatric combination vaccines. The quality control of these vaccines requires the determination of potency and the majority are now tested using an in vitro potency assay which measures the HBsAg content. Manufacturers have developed assays that are product specific but these are all mainly based on a commercial ELISA kit (Auszyme™ EIA kit from Abbott laboratories) which is recommended by the European Pharmacopoeia [1] and used by many national control laboratories for official testing. However the Auszyme™ kit is to be discontinued by Abbott laboratories and so we have initiated replacement of this kit by developing an alternative ELISA method.

The Auszyme™ kit is based on a sandwich technique using beads coated with mouse monoclonal IgM anti-HBsAg and it can be used on vaccines without prior desorption of the HBsAg from aluminium adjuvant. It was also planned to avoid pretreatment of samples in the new ELISA method. The initial approach was to develop a sandwich ELISA using monoclonal or polyclonal antibodies. However, problems were encountered with non-specific binding due to the fixation of vaccine adjuvant to micro plates. There were also significant differences in the HBsAg values obtained for different hepatitis B-containing combination vaccines which vary in adjuvant content. The only way to suppress these issues was to use an inhibition assay which indirectly measures the HBsAg content using human polyclonal antibodies thereby diminishing any interference due to the presence of adjuvant in the ELISA plate.

We have conducted validation studies on this new ELISA with a view to providing a replacement standard validated method which could be used by national control laboratories to release various HBsAg containing vaccines and thus avoid any interruption of vaccine supply that could arise due to the discontinuation of the Auszyme™ kit.

### 2. PARTICIPANTS

Most of the validation experiments were performed by one vaccine manufacturer while four Official Medicine Control Laboratories (OMCLs) participated in a collaborative study to assess reproducibility. These OMCLs were: the Scientific Institute of Public Health (ISP) from Belgium, the Sanitary Safety of Health Products Agency (AFSSAP) from France, the Paul Ehrlich Institute (PEI) from Germany and the National Institute for Biological Standard and Control (NIBSC) from the UK. A list of participants is provided at the end of this report in section 8.

### 3. MATERIAL AND METHODS

3.1. Vaccine Samples

All vaccines used in the study (see Table 1) were manufactured by GlaxoSmithKline (GSK) Biologicals, Rixensart, Belgium. In order to artificially reduce potency, some vaccines samples were stressed by incubation for 7 days or 15 days at 60° C. or by incubation overnight at 37° C. with 100 ppm of hydrogen peroxide. Following treatment, samples were stored at 4° C. until testing.

3.2. Reference Vaccine

The reference standard used for both the Auszyme™ method and the new inhibition ELISA is the European Pharmacopoeia Hepatitis B vaccine (rDNA) BRP2b reference. This reference contains 20 μg/ml of HBsAg as measured by the Lowry protein assay.

3.3. Human Polyclonal Antibodies.

A commercial preparation of human polyclonal anti-HBs antibodies (Nabi-HB™—Nabi Biopharmaceuticals, Boca Raton, Fla. USA) was used in the new inhibition ELISA method. In order to verify that this preparation contained antibodies that bind to a protective epitope of HBsAg, it was tested in a competition assay with RF1HBs monoclonal antibodies. RF1HBs antibodies have been shown to block infectivity of HBV in susceptible chimpanzees [2, 3].

Two fold dilutions of the test sample were performed in buffer containing a fixed amount (6.25 ng/ml) of biotinylated RF1 monoclonal antibodies (mAb) and incubated for 1.5 hours at 37° C. on HBsAg coated plates. After a washing step, the streptavidin-HRP Amdex (Amersham, Uppsala, Sweden) complex diluted in saturation buffer was added to the wells and incubated for 30 min at 37° C. Plates were then washed and incubated with a solution of tetramethylbenzidine substrate for 10 min at 22° C. The reaction was stopped with H₂SO₄. The optical density (OD) which is inversely proportional (competition assay) to the quantity of RF1-like antibody antibodies was measured by spectrophotometry (450 nm).

3.4. Determination of HBsAg Content Using New Inhibition ELISA

An overview of the method is provided in FIG. 1. Standard and vaccine samples were diluted with PBS containing 0.1% BSA and 0.1% Tween 20. 100 μl of each dilution was incubated, overnight at 25° C. under agitation, with 100 μl of a fixed amount of human polyclonal anti-HBs antibodies (±170 mIU anti-HBs/ml) (Nabi-HB™—Nabi Biopharmaceuticals, Boca Raton, Fla. USA) in Greiner Bio-One plates for cell culture (Greiner Bio-One, Frickenhausen, Germany). Plates were then centrifuged for 10 minutes at 2000 rpm and the supernatants transferred to ELISA plates (Maxisorp immuno-plate from NUNC, Roskilde, Denmark) coated overnight at 4° C. with 1 μg/ml HBsAg (from GSK) and post-coated with PBS-1% BSA. The plates were then incubated for 2 hours at 37° C., under agitation.

After washing, 100 μl of a 1/10000 dilution of goat anti-human IgG antibodies labelled with peroxidase (Sigma-Aldrich, St-Louis, Mo. USA) was added to each well. The plates were then incubated for 1 hour at 37° C. under agitation and then washed. The amount of bound antibodies was measured colorimetrically by adding tetramethylbenzidine/hydrogen peroxide solution as substrate. After 20 minutes incubation in the dark at room temperature, the reaction was stopped by addition of H₂SO₄, and OD read at 450 nm. The OD measured is inversely proportional to the amount of HBsAg in the unknown samples.

3.5. Determination of HBsAg Content Using Auszyme™ EIA Kit

This method uses the Abbott Auszyme™ ELISA kit (Abbott Laboratories, Chicago, Ill. USA), and was the European Pharmacopoeia method B for assay of hepatitis B vaccine (rDNA) [1].

Briefly, vaccines were diluted in PBS containing 0.2% BSA. Dilutions were incubated with plastic beads coated with monoclonal anti-HBs antibodies. After 3 hours incubation at 40° C., the beads were washed and incubated with monoclonal anti-HBs antibodies labelled with peroxidase. After 30 minutes incubation at room temperature, the beads were washed. The amount of bound HBsAg was measured colorimetrically by adding ortho-phenylene-diamine/hydrogen peroxide as a substrate. After stopping the reaction by addition of H₂SO₄, the OD was read at 490 nm. The OD measured is proportional to the amount of HBsAg in the unknown samples.

3.6. Calculation of In Vitro Potency

For both assays, the HBsAg content of test samples was calculated using the statistical method of parallel-line analysis [4]. Based on a series of dilutions a p value for the linearity of the sample dilutions and the parallelism of the response relative to the standard curve was determined for each potency result. A result was considered valid if the values of p for both linearity and parallelism were higher than 0.05. If the p values were below 0.05, the result was considered invalid and a repeat determination was performed.

3.7. Dose-Response Range

In routine practice, a dose-range curve containing 360, 300, 240, 180, 120 and 60 ng HBsAg/ml of the reference standard is established for each determination. Test samples are diluted accordingly.

The parallel-line analysis employed to calculate the potency values uses only the linear part of the curve dose/response. To show that the dose response curve for the reference standard and for the samples is linear between the highest and the lowest doses used in routine testing, an experiment was performed by progressively diluting the reference preparation and one lot of each of Engerix™-B, Twinrix™ and Infanrix™ penta vaccines (see Table 1). The dilutions were carried out in a two-fold serial manner to 1:1024 corresponding to a HBsAg concentration of 19.5 ng/ml for the reference. Each sample was tested in duplicate.

3.8. Limit of Quantification (LOQ)

As the OD values used for the calculation of the sample must be between the highest and the lowest OD values obtained for the reference standard, the lowest antigen concentration allowing a valid quantitation is the lower value determined for the range of quantification.

3.9. Limit of Detection (LOD)

Measurement of the magnitude of analytical background response was performed by analyzing the blank sample (PBS, Tween 0.1%, BSA 0.1%) six times in six independent experiments and calculating the mean OD values and the standard deviations. The average OD of the assay blank minus three standard deviations was calculated. This OD value was then introduced in the equation of the standard curve established with the reference, which allowed determination of the equivalent titre by regression analysis using the equation y=ax+b (y=optical density, x=log concentration, a=slope of the calibration curve, b=intercept). After correction for the dilution, the LOD value was calculated to obtain the analytical background response.

3.10. Linearity and Parallelism

The linear correlation coefficient of the six standard curves established with the reference standard to determine the LOD were used to determine linearity. In addition, as previously indicated, p values for linearity and the parallelism were calculated for each sample during the parallel-line analysis.

3.11. Precision

To assess the repeatability (intra-assay precision) three lots each of Engerix™-B, Twinrix™ and Infanrix™ penta vaccines were tested six times in the same experiment.

To assess the intermediate precision (inter-assay precision) three lots each of Engerix™-B, Twinrix™ and Infanrix™ penta vaccines were tested in six independent experiments performed by three technicians over four months. The validation was performed with two different lots of Nabi-HB™ to take into account the inter-lot variability of the reagent. Both lots had the same titre of anti-HBsAg and therefore were used at the same dilution.

To assess reproducibility (inter-laboratory precision) one lot each of Engerix™-B, Twinrix™ and Infanrix™ penta, was tested in parallel by our laboratory and four European OMCLs: Each laboratory was supplied with the Nabi-HB™, coating HBsAg and reference reagents and tested each vaccine lot in three independent sessions.

All precision results are expressed as coefficients of variation (standard deviation/mean×100).

3.12. Accuracy

The accuracy was assessed in a recovery experiment carried out by adding a known quantity of the reference to samples of Engerix™-B, Twinrix™ and Infanrix™ penta vaccines. The reference, assigned a value of 20 μg HBsAg/ml by the Lowry method, was used to spike the test samples with 5 μg, 10 μg and 15 μg of HBsAg (corresponding to 250 μl, 500 μl and 750 μl), respectively. In parallel, test samples were tested after the addition of 250 μl, 500 μl and 750 μl of diluent as controls. The percentage recovery was calculated was follows:

[Empirical value_{spiked}/(empirical value_{unspiked}+theoretical value of the spike)]×100.

3.13. Specificity

The specificity was assessed by testing placebos and vaccines without HBsAg. The non-specific binding of the Nabi-HB™ reagent to aluminium adjuvants (i.e. Al(OH)₃ and AlPO₄) was examined by testing placebos of Engerix™-B and Infanrix™ penta. Both placebos were tested neat, 1:2, 1:4, and 1:8.

To confirm the lack of cross-reactivity between the Nabi-HB™ reagent and other vaccine antigens, various vaccines which do not contain HBsAg (see Table 1) were tested neat, 1/10, 1/20 and 1/83 (lowest dilution used to test vaccines). Both placebos were also added in this experiment. In parallel, two lots of Engerix™-B vaccine were tested at a dilution of 1/83.

3.14. Bridging with the Auszyme™ Method

The HBsAg content was determined by both the new ELISA and Auszyme™ methods for lots of Engerix™-B, Twinrix™ and Infanrix™ penta vaccines. Results were compared by the paired t-test.

The ability of both methods to measure potency values at the lower and upper extremes of the normal commercial range was also assessed. This involved testing of vaccine lots with reduced potency induced by stress treatment (see section 2.1) and of lots with higher potency due to removal of thiomersal (development lots of candidate thiomersal free vaccines containing 10 to 20 μg/ml HBsAg) or increased antigen content (Fendrix™ see Table 1).

### 4. RESULTS

4.1. Robustness of Method

During assay development the following parameters were identified as critical to assay reliability. First, the presence of BSA in the dilution buffer is important to avoid non specific binding of antibodies to the adjuvant, during the overnight incubation. Second, during this overnight incubation, the agitation of the micro plate is also important to allow a correct interaction between the adsorbed Ag and antibodies. Finally, the centrifugation step after the overnight incubation is critical to avoid the presence of adjuvant in the ELISA plate. This centrifugation can be replaced by sedimentation of at least 30 minutes at room temperature (also validated, data not shown).

4.2. Characterization Human Polyclonal Antibodies (Nabi-HB™)

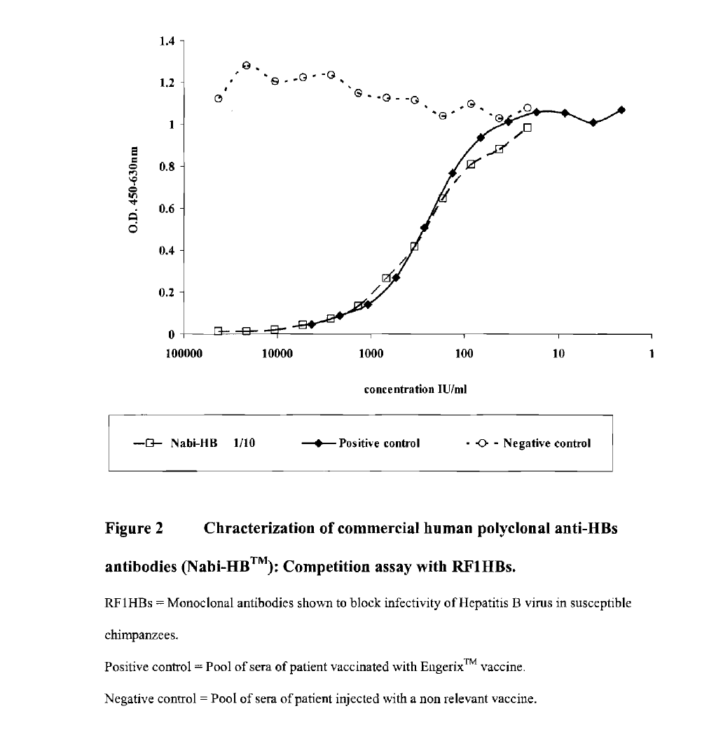

FIG. 2 shows the results of the competition assay against protective RF1 monoclonal antibodies for the Nabi-HB™ human polyclonal antibody solution compared to a positive control, (a pool of sera of patients vaccinated with Engerix™-B vaccine) and a negative control (pool of sera of patient injected with a non relevant vaccine). In this assay, anti-HBs antibodies present in the test samples compete with a fixed amount of biotinylated anti-RF1 mAbs for binding to HBsAg. The OD is therefore inversely proportional to the quantity of antibodies binding to the same epitope as the anti-RF1 mAbs. The results demonstrate that the level of RF1-like protective antibodies in the Nabi-HB™ preparation is similar to that found in the pool of sera from Engerix™-B vaccinees.

4.3. Dose-Response Range

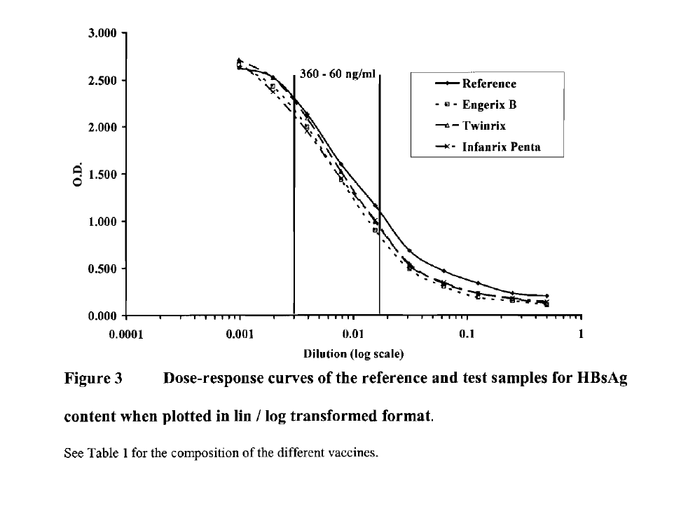

The dose-response curves shown in FIG. 3 reveal a characteristic sigmoidal shape when plotted in lin/log transformed format (OD on the Y-axis and inverse dilution on the log scale X-axis). The graph is linear for dilutions corresponding to HBsAg concentrations used in routine practice (i.e. 360 and 60 ng/ml for the standard preparation and 240 and 60 ng/ml for the test preparations

4.4. Limit of Quantification

As the OD values used for the calculation of the sample must be between the highest and the lowest OD values obtained for the reference standard (which corresponds to 60 and 360 ng/ml) the lowest antigen concentration allowing a valid quantitation is 60 ng/ml.

4.5. Limit of Detection

Table 2 shows that the LOD value estimated from six independent experiments was 35 ng/ml.

4.6. Linearity and Parallelism

As shown in Table 2 the values of the correlation coefficients of the linear regression for the six standard curves were are all above 0.99, thereby demonstrating linearity for the analytical range of 60 to 360 ng/ml. In addition, potency values were only considered valid if the values of p for both linearity and parallelism were higher than 0.05. Compliance with this validity criterion is usually more than 95% (unpublished data), in this present study all the potency values obtained were valid.

4.7. Repeatability and Intermediate Precision

The repeatability (intra-assay precision) for three lots each of Engerix™-B, Twinrix™ and Infanrix™ penta tested six times is presented in Table 3. The CV values for repeatability were all below 7%.

Table 4 presents the intermediate precision (inter-assay precision) for three lots each of Engerix™-B, Twinrix™ and Infanrix™ penta tested in six independent experiments. The CV values for intermediate precision were all below 10%.

4.8. Reproducibility

The results of a collaborative study on reproducibility between our laboratory and four OMCL are presented in Table 5. Some results particularly in one laboratory were invalid due to non compliance with validity criteria (mainly linearity and parallelism). The CV calculated from all valid results however was less than 10% for Engerix™-B and Twinrix™ and less than 15% for Infanrix™ penta demonstrating the transferability of the method to the four OMCLs.

4.9. Accuracy

Results of the accuracy assessment are given in Table 6. The percentage recovery values obtained ranged between 91% and 113%.

4.10. Specificity

Preliminary experiments showed that the observed OD values for the aluminium placebos of Engerix™-B and Infanrix™ penta at dilutions of 1:2, 1:4, and 1:8 were lower (the standard curve is inversely related to [HBsAg]) than typically observed for the assay blank. However, at the lowest dilution (1:83) used to test vaccines, non-specific binding was not observed (data not shown). This was confirmed by the placebo results obtained in another experiment (Table 7) where the OD values of placebo dilutions from 1:10 were shown to be similar to the blank. Therefore although the Nabi-HB™ reagent binds non-specifically to aluminium at high concentrations, non-specific binding was not observed at dilutions typically used to test vaccine samples.

Table 7 also presents data for vaccines not containing HBsAg. As expected, all the undiluted vaccine samples (except for Hiberix™ which was the only vaccine not adsorbed on aluminium salts) had mean OD values above those of the blank. However when diluted at least 1:10, there was no evidence of non-specific binding and/or cross-reactivity to non-HBsAg vaccine components.

4.11. Bridging with the Auszyme™ Method

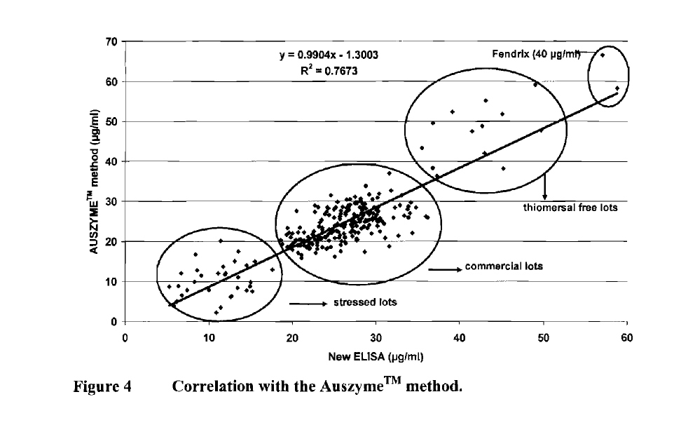

The potency results determined using both methods for different commercial lots of Engerix™-B (64 lots), Twinrix™ (69 lots) and Infanrix™ penta (62 lots) are presented in Table 8. Overall there was a good concordance between the methods for all three vaccines, but the new ELISA method yields, on average, higher results (significantly different for the three vaccines, as determined by a paired t-test, p<0.01). FIG. 4 presents the correlation between the two methods for the different types of vaccines tested including lots with reduced potency induced by stress treatment and lots with higher potency due to removal of thiomersal or higher antigen content (Fendrix™). The coefficient of correlation was 0.76 and there was a clear distinction between the sub-potent, normal range potency and high potency lots.

### 5. DISCUSSION

The discontinuation of the Auszyme™ kit used by manufacturers and national control labs to determine the HBsAg content of hepatitis B vaccines has led us to develop an alternative inhibition ELISA method. The ELISA was designed to allow the testing of vaccines without prior desorption of the antigen from the adjuvant, and to be applicable to different combination vaccines using the same BRP reference as previously employed.

The objective of this study was to demonstrate that the proposed ELISA was suitable for its intended purpose. First it was important to show that the quantitation of HBsAg by the assay was a relevant measure of vaccine potency. We established this by demonstrating that the commercial preparation of human polyclonal anti-HBs antibodies (Nabi-HB™) used in the assay contained antibodies that bind to a protective epitope of HBsAg and that the level of protective antibodies was similar to that found in sera from Hepatitis B vaccinees.

Subsequently we conducted validation studies on the assay according to The International Conference of Harmonization [5]. The performance characteristics of the ELISA were found to be satisfactory for all the hepatitis B-containing vaccines tested. The repeatability, intermediate precision and accuracy of the ELISA are similar to that of the previous method based on the Auszyme™ kit [1]. The sensitivity of the proposed ELISA is lower (LOQ of 60 ng/ml versus 2.5 ng/ml for the Auszyme™ method) but as most vaccines contain 10-20 μg/ml HBsAg this has no impact on the intended use of the assay. In fact the lower sensitivity may confer an advantage by reducing the number of dilution steps required and thereby minimizing potential error and variability.

We have shown that the proposed ELISA can specifically measure HBsAg in the presence of other vaccine antigens, this is important as HBsAg is increasingly employed as a component of combination vaccines. We have also demonstrated that although there is non specific binding of the Nabi-HB™ reagent to aluminium this does not interfere in the dilution range used in the assay.

A bridging study with the Auszyme™ method conducted with over 60 lots each of Engerix™-B Twinrix™ and Infanrix™ penta showed a good concordance between the two methods although the proposed ELISA method on average yielded higher results. For some vaccines this difference may have to be taken into consideration by a revision of the release specification. The correlation between the two methods also allowed identification of sub-potent lots and high potency lots (including thiomersal free lots, it has been observed (unpublished data) that removal of thiomersal from hepatitis B vaccine formulations results in an increase in potency).

Parallel testing in four Official Medicine Control Laboratories showed that the method can be easily transferred to other laboratories. No training was provided to the participating laboratories that were just supplied with the test protocol plus the Nabi-HB™, coating antigen and reference reagents. As a result there were slightly more invalid results observed but further experience and fine tuning of parameters should reduce these. Preliminary testing performed by some of the participating laboratories on vaccines from other manufacturers was promising. The results (unpublished data) indicated that the test could potentially be adapted for use with vaccines from other manufacturers by using homologous references and by altering the quantification range.

The successful reproduction of the proposed ELISA which uses readily available reagents and its potential to be applied to vaccines from other manufacturers indicates that it could provide the basis for a common method which could be used worldwide by national control laboratories and authorities.

### 6. CONCLUSION

The proposed ELISA is suitable to measure the HBsAg content of different hepatitis B vaccines and provides a reliable alternative to the method using the discontinued Auszyme™ kit.

### 7. REFERENCES

[1] Assay of hepatitis B vaccine (rDNA), general chapter 2.7.15. Ph. Eur. 5^{th} edition. Strasbourg France Council of Europe; 2005.
[2] Iwarson S, Tabor E, Thomas H C et al. Neutralisation of hepatitis B virus infectivity by a murine monoclonal antibody: an experimental study in the chimpanzee. J Med Virol 1985; 16: 89-95.
[3] Waters J A, Brown S E, Steward M W et al. Analysis of the antigenic epitopes of hepatitis B surface antigen involved in the induction of a protective antibody response. Virus research 1991; 22:1-22.
[4] Finney D J. Statistical Method in Biologicals Assay, chapter 4, third edition. London: Griffin, 1978.
[5] Validation of analytical procedures: methodology. Ref Q2B ICH; 1996. November [guideline].

TABLE 1
Summary of the composition of vaccines used in the study.

| | HBsAg | | |
|---|---|---|---|
| Vaccine | Content* | Other Antigens | Adjuvant |
| Engerix ™- | 20 μg/ml | — | Aluminum hydroxide |
| Twinrix ™ | 20 μg/ml | Inactivated Hepatitis A virus | Aluminum hydroxide + |
| | | | Aluminum |
| | | | phosphate |
| Fendrix ™ | 40 μg/ml | — | AS04 ™ adjuvant + |
| | | | Aluminum phosphate |
| Infanrix ™ | 20 μg/ml | Diphtheria toxoid | Aluminium hydroxide + |
| penta | (actual dose 10 μg | Tetanus toxoid | Aluminum |
| (also | as vaccine | Pertussis toxoid | phosphate |
| known as | presented as | Filamentous haemagglutinin | |
| Pediarix ®) | 0.5 ml) | Pertactin | |
| | | Poliovirus type 1 (Mahoney) | |
| | | Poliovirus type 2 (MEF-1) | |
| | | Poliovirus type 3 (Saukett) | |
| Infanrix ™ | — | Same as Infanrix ™ penta | Aluminum hydroxide + |
| IPV | | | Aluminum |
| | | | phosphate |
| Infanrix ™ | — | Diphtheria toxoid | Aluminum hydroxide + |
| | | Tetanus toxoid | Aluminum |
| | | Pertussis toxoid | phosphate |
| | | Filamentous haemagglutinin | |
| | | Pertactin | |
| Havrix ™ | — | Inactivated Hepatitis A virus | Aluminium hydroxide |
| Tritranrix ™ | — | Diphtheria toxoid | Aluminum phosphate |
| | | Tetanus toxoid | |
| | | Whole cell B. pertussis antigens | |
| Hiberix ™ | — | H. influenzae type b polyribosyl | — |
| | | ribitol phosphate conjugated to | |
| | | tetanus toxoid | |

*as determined by Lowry protein assay

TABLE 2
Limit of detection (LOD)

| | Optical density | | | | | | |
|---|---|---|---|---|---|---|---|
| | (OD) - Blank¹ | | | | | | |
| | | Standard | | Y | | | LOD⁵ |
| Date | Mean² | deviation² | Slope³ | intercept | (R²)⁴ | X⁵ | (ng/ml) |
| 29/04/2004 | 3.140 | 0.026 | −1.9678 | 6.0192 | 0.9968 | 1.502795 | 31.8 |
| 18/06/2004 | 2.870 | 0.058 | −1.6569 | 4.9266 | 0.9973 | 1.346249 | 22.2 |
| 06/05/2004 | 3.096 | 0.061 | −1.9811 | 6.0878 | 0.9986 | 1.602544 | 40.0 |
| 08/07/2004 | 3.332 | 0.047 | −2.0408 | 6.5585 | 0.9974 | 1.650088 | 44.7 |
| 16/07/2004 | 3.278 | 0.022 | −2.0834 | 6.4546 | 0.9980 | 1.556398 | 36.0 |
| 04/05/2004 | 3.347 | 0.049 | −2.0391 | 6.2911 | 0.9992 | 1.515914 | 32.8 |
| | | | | | | Mean | 34.6 |

¹The blank sample is the dilution buffer (PBS, Tween 0.1%, BSA 0.1%).
²The magnitude and variation of analytical background response is performed by analyzing six times the blank sample.
³Slope of the calibration curve.
⁴R² is a measure of goodness-of-fit of linear regression.
⁵LOD was calculated by taking into consideration the average OD of the blank signal minus three standard deviations. This OD value was then introduced in the equation of the standard curve established with the reference standard, which allowed determination of the equivalent titre by regression analysis using the equation y = ax + b, where y = OD − (3 * st. dev.), x = concentration, a = slope of the calibration curve, b = intercept). After correction for the dilution, the LOD value was calculated to obtain the analytical background response.

TABLE 3
Repeatability (Intra-assay precision).

| | Mean² | | |
|---|---|---|---|
| | HBsAg | | |
| | content | Standard | Coefficient of |
| Vaccine¹ | (μg/ml) | deviation | variation (%)³ |
| Engerix ™-B | | | |
| Lot 1 | 22.5 | 0.6 | 2.7 |
| Lot 2 | 28.8 | 1.4 | 5.0 |
| Lot 3 | 26.5 | 1.6 | 5.9 |
| Twinrix ™ | | | |
| Lot 1 | 25.0 | 1.2 | 4.9 |
| Lot 2 | 25.6 | 1.3 | 4.9 |
| Lot 3 | 28.7 | 1.0 | 3.6 |
| Infanrix Penta ™ | | | |
| Lot 1 | 25.9 | 1.3 | 4.9 |
| Lot 2 | 30.0 | 0.9 | 3.0 |
| Lot 3 | 23.6 | 1.5 | 6.4 |

¹See Table 1 for the composition of the different vaccines.
²Each lot was tested six times on the same day.
³Coefficient of variation = (standard deviation/mean) * 100.

TABLE 4
Intermediate precision (Inter-assay precision)

| | Mean² | | |
|---|---|---|---|
| | HBsAg | | |
| | content | Standard | Coefficient of |
| Vaccine¹ | (μg/ml) | deviation | variation (%)³ |
| Engerix ™-B | | | |
| Lot 1 | 22.0 | 0.9 | 4.0 |
| Lot 2 | 27.1 | 1.5 | 5.6 |
| Lot 3 | 28.2 | 0.9 | 3.2 |
| Twinrix ™ | | | |
| Lot 1 | 25.2 | 0.9 | 3.5 |
| Lot 2 | 26.8 | 0.9 | 3.4 |
| Lot 3 | 27.2 | 2.2 | 8.0 |
| Infanrix Penta ™ | | | |
| Lot 1 | 26.2 | 2.4 | 9.3 |
| Lot 2 | 31.1 | 2.4 | 7.7 |
| Lot 3 | 25.2 | 2.3 | 9.1 |

¹See Table 1 for the composition of the different vaccines.
²Each lot was tested six independent experiments.
³Coefficient of variation = (standard deviation/mean) * 100.

TABLE 5
Reproducibility in four Official Medicines Control Laboratories
(OMCLs).

| Labora- | | HBsAg content (μg/ml) | | |
|---|---|---|---|---|
| tory | Testing | Engerix ™-B¹ | Twinrix ™¹ | Infanrix ™ Penta¹ |
| GSK | 1 | 25 | 23.9 | 21.7 |
| | 2 | 26.2 | 23.5 | 20.6 |
| | 3 | 24.5 | 23.6 | 22.5 |
| OMCL 1 | 1 | 29 | inv² | inv² |
| | 2 | 27.1 | 23.3 | 22 |
| | 3 | 27.1 | 22.5 | 21.2 |
| OMCL 2 | 1 | 24.8 | 18.1 | 18.5 |
| | 2 | 24.6 | 21.1 | 15.8 |
| | 3 | 28.2 | 22.9 | 18.9 |
| OMCL 3 | 1 | 27.3 | 22.5 | 24.6 |
| | 2 | 28.7 | 23.1 | 25.3 |
| | 3 | 28.7 | inv² | 21.7 |
| OMCL 4 | 1 | 30.7 | 23.9 | 22.7 |
| | 2 | inv² | inv² | inv² |
| | 3 | inv² | inv² | 24.2 |
| | Mean | 27.1 | 22.6 | 21.5 |
| | SD | 2.0 | 1.7 | 2.6 |
| | CV (%) | 7.3 | 7.5 | 12.2 |

¹See Table 1 for the composition of the different vaccines.
²Invalid results due to non compliance with validity criteria.

TABLE 6
Accuracy

| 1. Spiked | | | |
|---|---|---|---|
| quantity | | | |
| 2. (μg HBsAg)¹ | 1. Recovery (%)² | | |
| 3. | 2. Engerix ™-B³ | 1. Twinrix ™³ | 1. Infanrix |
| | | | Penta ™³ |
| 4. 5 μg | 3. 112% | 2. 105% | 2. 100% |
| 5. 10 μg | 4. 107% | 3. 113% | 3. 106% |
| 6. 15 μg | 5. 104% | 4. 91% | 4. 95% |

¹The reference has been given a value of 20 μg HBsAg/ml (labelled value) as measured by the Lowry method.
²Recovery (%) = [Empirical values_{spiked}/(empirical values_{unspiked} + theoretical value of the spike)] × 100.
³See Table 1 for the composition of the different vaccines.

TABLE 7
Specificity.

| | OD value | | | |
|---|---|---|---|---|
| | Dilution | | Dilution | Dilution |
| Test samples | 1/83 | Undiluted | 1/10 | 1/20 |
| Placebo | | | | |
| AlPO₄¹ | 2.959 | 0.370 | 2.875 | 2.884 |
| Al(OH)₃² | 2.824 | 1.41 | 2.830 | 2.931 |
| Vaccine without HBsAg³ | | | | |
| Havrix ™ | 3.009 | 1.397 | 2.934 | 2.971 |
| Tritranrix ™ | 2.860 | 0.572 | 2.867 | 2.929 |
| Infanrix ™ IPV | 2.823 | 0.568 | 2.917 | 2.909 |
| Infanrix ™ | 2.832 | 0.656 | 2.910 | 2.967 |
| Hiberix ™ | 2.810 | 2.926 | 3.005 | 3.023 |
| Vaccine containing HBsAg³ | | | | |
| Engerix ™-B lot 1 | 0.991 | — | — | — |
| Engerix ™-B lot 2 | 1.000 | — | — | — |
| Blank | | | | |
| Dilution buffer | 2.856 | — | 2.860 | — |

¹Placebo representing Infanrix Penta ™
²Placebo representing Engerix ™-B
³See Table 1 for the composition of the different vaccines

TABLE 8
Bridging with the Auszyme ™ method.

| | | |
|---|---|---|
| 1. | 1. Potency (μg/ml) | |
| 2. | 2. New ELISA | 1. Auszyme ™ |
| | | method |
| 3. Engerix ™-B | 3. | 2. |
| 4. n | 4. 64 | 3. 64 |
| 5. Mean | 5. 25.3 | 4. 24.4 |
| 6. Standard | 6. 3.6 | 5. 3.5 |
| deviation | | |
| 7. CV (%) | 7. 14.2 | 6. 14.3 |
| 8. Twinrix ™ | 8. | 7. |
| 9. n | 9. 69 | 8. 69 |
| 10. Mean | 10. 28.9 | 9. 27.0 |
| 11. Standard | 11.3.4 | 10.2.7 |
| deviation | | |
| 12. CV (%) | 12. 11.7 | 11. 9.9 |
| 13. Infanrix | 13. | 12. |
| Penta ™ | | |
| 14. n | 14. 62 | 13. 62 |
| 15. Mean | 15. 25.5 | 14. 21.6 |
| 16. Standard | 16. 3.6 | 15. 3.1 |
| deviation | | |
| 17. CV (%) | 17. 14.2 | 16. 14.4 |

CV⁼ Coefficient of variation = (standard deviation/mean) * 100.

1. A method for at least one of detection and quantification of an antigen linked to a metal salt, the method comprising:
(i) contacting the antigen with an antibody specific for that antigen;
(ii) contacting any unbound antibody with an antigen linked to a solid support, under conditions that allow any antibody present to form an antibody-antigen complex with the antigen on the solid support; and
(iii) detecting the antibody-antigen complex formed on the solid support.

2. The method of claim 1, wherein the antigen is a hepatitis B surface antigen linked to an aluminium salt.

3. The method of claim 1, wherein the antigen linked to a metal salt is in combination with at least one additional antigen.

4. The method of claim 3, wherein the at least one additional antigen is selected from the group of:
Hepatitis A antigen, Diphtheria toxoid, Tetanus toxoid, Pertussis toxoid, Whole cell pertussis antigen; Filamentous haemagglutinin, Pertactin, Poliovirus type; H. influenzae type b polyribosyl ribitol phosphate.

5. The method of claim 1, wherein any unbound antibody present at the end of step (i) is purified away from the bound antibody-antigen prior to step 2.

6. A kit for detection, quantification, or detection and quantification of an antigen linked to a metal salt, the kit comprising instructions to carry out the method of the invention and at least one other component selected from:
1 an antibody (detection antibody) specific for an antigen to be detected;
2 an antigen, optionally linked to a solid support;
3 detection reagents for detection of any antibody-antigen complex formed on the solid support;
4 positive control; and
5 negative control.

7. The method of claim 4, wherein the at least one additional antigen comprises Hepatitis A antigen.

8. The method of claim 4, wherein the at least one additional antigen comprises Diphtheria toxoid, Tetanus toxoid, Pertussis toxoid, Filamentous haemagglutinin, Pertactin, Poliovirus type 1, Poliovirus type 2, and Poliovirus type 3.

9. The method of claim 4, wherein the at least one additional antigen comprises Diphtheria toxoid, Tetanus toxoid, Pertussis toxoid, Filamentous haemagglutinin, and Pertactin.

10. The method of claim 4, wherein the at least one additional antigen comprises Diphtheria toxoid, Tetanus toxoid, and whole cell pertussis antigens.

11. The method of claim 4, wherein the at least one additional antigen comprises H. influenzae type b polyribosyl ribitol phosphate conjugated to tetanus toxoid.
